(19) United States
(12) Patent Application Publication
(10) Pub. No.: US 2020/0215286 A1
(43) Pub. Date: Jul. 9, 2020

(54) Expandable Endotracheal Tube

(71) Applicants: Fanping Wang, Encino, CA (US); Kongyuan HE, Encino, CA (US)

(72) Inventors: Fanping Wang, Encino, CA (US); Kongyuan HE, Encino, CA (US)

(21) Appl. No.: 16/825,640

(22) Filed: Mar. 20, 2020

Related U.S. Application Data
(60) Provisional application No. 62/960,539, filed on Jan. 13, 2020.

(51) Int. Cl. A61M 16/04

(52) U.S. Cl. CPC A61M 16/0488; A61M 25/0102; A61M 16/0434

(57) ABSTRACT

The present invention is directed to a novel endotracheal tube assembly comprising an endotracheal tube and an expander. The endotracheal tube of the present invention comprises a tunnel configured into the wall of the endotracheal tube, wherein the tunnel can be expanded to increase the circumference of the endotracheal tube. The tunnel can replaceably receive the expander of different widths, thus gradually increasing the circumference, and thus the internal diameter, of the endotracheal tube, without removing the endotracheal tube from the trachea.

### CROSS-REFERENCE TO RELATED APPLICATIONS

This application claims priority to the U.S. provisional patent application Ser. No. 62/960,539 filed Jan. 13, 2020, entitled “Controlled Expandable Diameter of endotracheal tube (CED-ETT)”, which is incorporated herein by reference in its entirety.

### FIELD OF INVENTION

The present invention relates to medical devices, and more particularly, to airway devices, such as tracheal tubes.

### BACKGROUND

An endotracheal tube is a medical device that is inserted through the mouth into the trachea of a patient for the primary purpose of establishing and maintaining a patent airway. The Endotracheal tube also referred hereinafter as a tube, is a flexible plastic tube that is frequently used for airway management in the settings of emergency medicine, critical care, mechanical ventilation, and surgery with anesthesia. The tube is a commonly used medical device in critical patient care. The procedure for inserting the tube into the trachea is known as endotracheal intubation.

Endotracheal tubes are provided in different sizes ranging from 2.0 millimeters to 12.0 mm internal diameters in 0.5 mm increments. In general, a 7.0 to 7.5 mm diameter tube is often used for females and an 8.0 to 9.0 mm diameter tube for men. Newborns often require a 3.0 mm to 3.5 mm tube, with a 2.5 to 3.0 mm tube used for premature infants.

It is important to have the correct size of the tube for a patient. Different sizes of the tube can have both advantages and disadvantages. The advantages of smaller size tubes include easier insertion, lower incidence of sore throats after intubation. Thus, it can be said that tubes having a smaller diameter is safer than the large-sized tubes. The trachea is a very sensitive organ of the body and the tube of the smaller size can relatively easily be inserted into the trachea. Moreover, the view of the larynx during the passage of the tube is subjectively better and the intubation is likely to be less traumatic than a larger size tube. However, there are also disadvantages of smaller size tubes. A small diameter tube is more likely to be obstructed by secretions. The entry of secretions into a small diameter tube is likely to cause a large increase in resistance and cause obstruction to gas flow. The narrower the tube, the greater is the resistance to gas flow. Also, bulky secretions require suction catheters for efficacy. But, in smaller endotracheal tubes, the catheter may obstruct a significant portion of the tube. During bronchoscopy, with smaller endotracheal tubes, the bronchoscope can be difficult to insert. The resistance to flow in the airway depends on the dimensions of the airway. With greater airway resistance, the gas pressure delivered to the alveoli is reduced.

The larger size tubes have certain advantages over small-sized tubes. Large size tubes can permit other devices to pass through the tube more easily than a small size tube. For example, larger diameter suction tubes or bronchoscopes can be more easily inserted into large-sized tubes. Furthermore, driving pressures in larger tubes can be reduced.

Therefore, doctors select the appropriate size of the tube for a patient. In the process of intubation, the size of endotracheal intubation may affect the success of the operation. Also, depending upon the condition of the patient, the doctors may use a larger or smaller size of the tube than will normally apply to the patient. Doctors may often select a larger size of tube than is normally appropriate for the patient. This is critically important to the spontaneously breathing patient who have to work harder to overcome the increased resistance. Positive pressure ventilation will usually need to be converted to spontaneous ventilation before intubation.

Doctors often guess at the right size, while in the operating room the size is often chosen based on age and body weight. However, the guess is not always right, and the inserted tube has to be replaced with a proper size tube. Often the change in the size of the tube is mandated based on the change in the medical condition of the patient. Replacing the tube is often a complex procedure and may be life-threatening with morbidity. For example, it may lead to esophageal intubation, loss of the airway, severe hypoxia, or cardiac arrest. The risk may be greatest in the patient with a difficult airway or those with poor cardiopulmonary reserve regarding underlying disorders. In the critically ill patient, an important practical consideration is that once a tube is placed, replacing it is often hazardous.

Thus, a need is appreciated for an improved endotracheal tube of which the internal diameter can be varied in a controlled manner. The need is appreciated for an improved endotracheal tube of which the internal diameter can be adjusted according to the requirement of the patient, avoiding replacing the endotracheal tubes and consequences related to the procedure of replacing the endotracheal tube. A need is appreciated for an endotracheal tube of which the internal diameter can be strictly and accurately controlled in each millimeter unit, which may avoid the procedure of replacing the endotracheal tube and morbidity, mortality, and cost involved with the replacement of the endotracheal tube.

### SUMMARY OF THE INVENTION

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The principal objective of the present invention is therefore directed to an improved endotracheal tube assembly having an endotracheal tube of which the internal diameter can be gradually increased.

It is another objective of the present invention that the need to replace the endotracheal tube with a larger size endotracheal tube is avoided.

It is yet another objective of the present invention that the endotracheal tube assembly is economic to manufacture.

In one aspect, the present invention is directed to a novel endotracheal tube assembly comprising an endotracheal tube and an expander. The endotracheal tube of the present invention comprises a tubular body having a proximal end, a distal end and a wall extending between the proximal end and the distal end. An opening of the endotracheal tube at the proximal end can be fluidly connected to a medical apparatus, while the distal end is received into the trachea. One or more tunnels configured into the wall of the tubular body, wherein the one or more tunnels extend between the proximal end and the distal end along the length of the wall. The one or more tunnels configured to be expanded in width, thus increasing the internal diameter of the endotracheal tube. The tunnel is open at the proximal end and closed at the distal end.

In another aspect, the expander is having an arc shaped elongated body with one end configured as a handle and the other end configured as a blunted tip. The expander is made of a flexible material and configured to be received into the tunnel, wherein the blunted tip is inserted into the tunnel. The expander can be provided in different widths wherein the width of the expander is proportional to change in the internal diameter of the endotracheal tube.

In one aspect, the present invention provides a method for inserting the endotracheal tube through the mouth into the trachea of a patient. The method comprises the step of inserting the endotracheal tube into the trachea of the patient, wherein the tube is inserted through the distal end. After inserting the endotracheal tube, the expander of first width can be inserted through the opening of the tunnel at the proximal end into the tunnel, wherein the internal diameter of the endotracheal tube increases proportionally to the first width of the expander.

In one aspect, the present invention provides a method of increasing or decreasing the internal diameter of the endotracheal tube. The method comprising the step of inserting the endotracheal tube into the trachea of a patient, wherein the endotracheal tube is inserted at its distal end. The expander of first width can be inserted into the tunnel through the opening of the tunnel increasing the internal diameter of the endotracheal tube, wherein the increase in the internal diameter of the endotracheal tube is proportional to the first width. To further adjust the internal diameter of the endotracheal tube, the expander of first width can be removed from the tunnel and another expander of a second width, wherein the second width can be less than or greater than the first width, can be inserted into the trachea, and wherein the internal diameter of the endotracheal tube is proportional to the second width.

These and other objects and advantages of the embodiments herein will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a side view of the endotracheal tube, in accordance with an embodiment of the present invention.

FIG. 2 shows an opening of the endotracheal tube and a tunnel, in accordance with an embodiment of the present invention.

FIG. 3 is a bottom view of the endotracheal tube expander, in accordance with an embodiment of the present invention.

FIG. 4 shows the opening of the endotracheal tube of FIG. 2 having the endotracheal tube expander inserted into the tunnel, in accordance with an embodiment of the present invention.

Although the specific features of the present invention are shown in some drawings and not in others. This is done for convenience only as each feature may be combined with any or all of the other features in accordance with the embodiments herein.

### DETAILED DESCRIPTION

In the following detailed description, a reference is made to the accompanying drawings that form a part hereof, and in which the specific embodiments that may be practiced is shown by way of illustration. These embodiments are described in enough detail to enable those skilled in the art to practice the embodiments and it is to be understood that the logical, mechanical, and other changes may be made without departing from the scope of the embodiments. The following detailed description is therefore not to be taken in a limiting sense.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

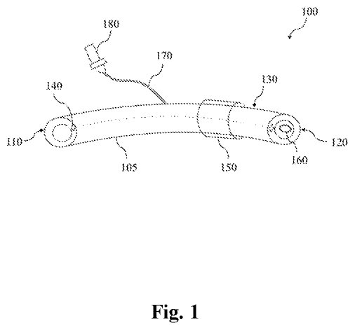

The various embodiments herein provide a novel endotracheal tube assembly having a novel endotracheal tube and an expander. Referring to FIG. 1, which shows an embodiment of the endotracheal tube 100 having a tubular body 105, the tubular body having a proximal end 110, a distal end 120, and a wall 130 that extends between the proximal end 110 and the distal end 120. The endotracheal tube 100 can be inserted through its distal end 120 into the trachea, while the proximal end 110 can be connected to a medical apparatus. A tunnel 140 can be configured into the wall 130 wherein the tunnel 140 extends between the proximal end 110 and the distal end 130, along the length of the wall 130. The tunnel 140 can be open at the proximal end and sealed at the distal end 120. While FIG. 1 is having one tunnel, more than one tunnel can be configured into the wall 130 of the endotracheal tube 100. For example, two tunnels can be configured at opposite ends of the diameter of the endotracheal tube. FIG. 1 further shows a cuff 150 circumscribed over the tubular body 105. The distal end 120 of the tubular body 105 is configured as a beveled atraumatic tip and a murphy eye 160 is configured near the distal end 120.

In one embodiment, the endotracheal tube 100 can be made of any medically approved plastic material that is flexible. The endotracheal tube 100 can be sufficiently flexible to follow the contour of the patient's trachea without requiring excessive force and to accommodate variations in the size and anatomy of the trachea. However, the endotracheal tube 100 can also have sufficient rigidity to be advanced into the patient's trachea without buckling. Preferably, the endotracheal tube can be made of polyvinyl chloride. However, any other material, such as silicone, rubber, and latex that is known to a skilled person for use in the manufacture of endotracheal tubes are within the scope of the present invention. The endotracheal tube 100 according to the present invention can be manufactured in different sizes, where a doctor can select the endotracheal tube of suitable size and can then gradually increase the size of the endotracheal tube within the pre-determined limits, while the endotracheal tube is into the trachea. Preferably, the doctor can determine the applicable size of the endotracheal tube for a patient, and then choose the endotracheal tube of a smaller size than the determined size, wherein the smaller size endotracheal tube can be inserted into the trachea and the size of the endotracheal tube can then be increased gradually till the desired size of the endotracheal tube is achieved.

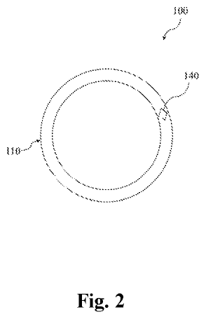

In FIG. 2 lumen of the endotracheal tube 100 can be seen within the wall 130. FIG. 2 shows the opening of the endotracheal tube at the proximal end 110. The opening of the endotracheal tube 100 at the proximal end 110 can be fluidly connected to a medical apparatus, for example, a breathing medical device. A connector, also known as an adaptor, can be used to connect the endotracheal tube 100 to the medical device. The connectors are generally available in standard sizes and the proximal end 110 of the endotracheal tube 100 can be configured to be coupled with commercially available connectors of standard sizes. It will be appreciated that the proximal end of the endotracheal tube 100 can be configured to be coupled with a universal 15 mm connector.

The distal end 120 of the endotracheal tube 100 shown in FIG. 1 can be inserted into the trachea of a patient. Preferably, the distal end of the endotracheal tube 100 is beveled and can be made of a material that is soft enough to be atraumatic. The beveled tip facilitates the insertion of the endotracheal tube 100 between the vocal cords. Further shown in FIG. 1 is a murphy eye 160 configured in the wall 130 immediately adjacent to the distal end 120. Although FIG. 1 shows one murphy eye, more than one murphy eye can be configured into the endotracheal tube. For example, one or two murphy eyes in a close relationship can be configured adjacent to the distal end 120 of the endotracheal tube 100. The functioning of the murphy eye is known to a skilled person for preventing the complete obstruction of the patient's airway, in case the distal opening of an endotracheal tube become occluded.

The cuff 150 circumscribes the exterior surface of the tubular body 105 near its distal end 120 so that the airway surrounding the endotracheal tube 100 can be occluded at the time the patient is connected to a ventilator, thereby allowing the ventilator to completely regulate the patient's respiration. The cuff 150 can be inflated and deflated by a separate mechanism attached to the cuff 150. Generally, an inflation tube 170 can be connected to the cuff 150 and the other end of the inflation tube can be connected to an inflation port 180. While FIG. 1 shows the endotracheal tube as having a cuff, endotracheal tube without a cuff is within the scope of the present invention.

FIG. 2 show an opening of tunnel 140, the tunnel is shown by a dashed line along the length of the tubular body 105. The tunnel 140 if of stretchable and folded construction that interrupts the wall 130. The body of the tunnel 140 can be coupled to the interrupted sides of the wall 130, such that expansion of the width of the tunnel 140 increases the circumference of the endotracheal tube 100. An inner layer or outer layer of the tunnel 140 can be covered and sealed with a flexible and expandable material (flexible thin-wall film), it can stretch, shrink; and is very thin. The layers of the tunnel 140 can be folded as well as stored inside the tunnel before the tunnel is opened. The inner layer of the tunnel is covered completely by the flexible thin-wall film, and, the outer side of the tunnel is covered completely or partially above the cuff to prevent air leakage. The internal lumen of the tunnel 140 can be round, elliptic or rhomboid.

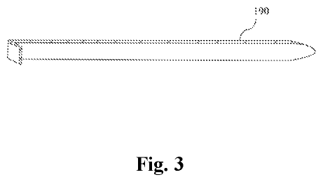

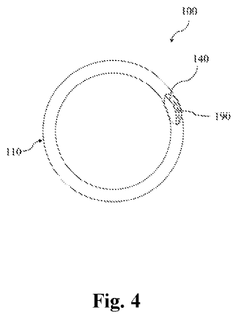

The opening of the tunnel at the proximal end 110 is temporarily closed when the endotracheal tube 100 is inserted into the trachea. After insertion of the endotracheal tube 100 into the trachea, the opening of the tunnel 140 can be opened at the proximal end 110 for insertion of an expander 190. An embodiment of the expander is shown in FIG. 3 having an arc or flat elongated body with one end configured as a handle and the other end configured as a blunted tip. The expander 190 is made of a flexible material and configured to be received into the tunnel 140, wherein the blunted tip is inserted into the tunnel 140. The expander 190 can be provided in different widths wherein the width of the expander 190 is proportional to change in the internal diameter of the endotracheal tube 100. FIG. 4 shows the proximal end 110 of the endotracheal tube 100 retaining the expander 190 into the tunnel 140. The expander 190 can be made of a medical-grade plastic material that is flexible similar to the endotracheal tube 100, such that the flexibility of the endotracheal tube 100 is not affected by the expander 190 retained into the tunnel 140. However, the expander 140 can be made of material enough rigid to allow the expander 190 to be inserted into the tunnel 140 without buckling.

As seen in FIG. 4, the expander 190 when inserted into the tunnel 140 causes expansion of the tunnel 140, such that the walls 130 of the tubular body 105 are expanded at the interrupted sides of the wall 130. This causes an increase in the circumference of the tubular body 105 and thus increasing the internal diameter of the endotracheal tube 100. It will be appreciated that an increase in width of the tunnel 140 is proportional to the width of the expander 190, thus expander 190 can be manufactured in different sizes.

In one case, the expander 190 can be inserted and removed from the endotracheal tube 100 without removing the endotracheal tube 100 from the trachea. And also, without removing the endotracheal tube 100 from the trachea, another expander of a different width can be inserted into the tunnel 140, thus changing the circumference of the endotracheal tube 100. In one case, after inserting the endotracheal tube 100, the expander 190 of a first width can be inserted through the opening of the tunnel 140 at the proximal end into the tunnel 140, wherein the internal diameter of the endotracheal tube 100 increases proportionally to the first width of the expander 190. In another case, the expander 190 of a first width can be inserted into the tunnel 140 through the opening of the tunnel 140 increasing the internal diameter of the endotracheal tube, wherein the increase in the internal diameter of the endotracheal tube is proportional to the first width. To further adjust the internal diameter of the endotracheal tube, the expander 190 of first width can be removed from the tunnel 140 and another expander of a second width, wherein the second width can be less than or greater than the first width, can be inserted into the trachea, and wherein the new internal diameter of the endotracheal tube 100 is proportional to the second width. Thus, the present invention allows a gradual increase in the internal diameter of the endotracheal tube without replacing the endotracheal tube. In one case, a doctor can choose an endotracheal tube of a smaller diameter than normally applicable for a patient. The smaller size endotracheal tube can be easily inserted into the trachea compared to a larger size endotracheal tube. Thereafter, the internal diameter can be gradually increased by using the expanders of increasing widths, till the desired internal diameter of the endotracheal tube is achieved.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An endotracheal tube assembly comprising:
an endotracheal tube having a proximal end, a distal end, and a wall extending between the proximal end and the distal end;
a tunnel extending between the proximal end and the distal end of the endotracheal tube and interrupting the wall of the endotracheal tube, the tunnel configured to be expanded to increase the circumference of the endotracheal tube; and
an expander having an elongated body, the expander configured to be removably received and retained into the tunnel, the expander configured to expand the tunnel, wherein a width of the expander is proportional to an increase in the circumference of the endotracheal tube.

2. The endotracheal tube assembly of claim 1, wherein the proximal end of the endotracheal tube can be fluidly connected to a medical device and the distal end of the endotracheal tube can be configured to be inserted into a trachea.

3. The endotracheal tube assembly of claim 2, wherein the proximal end is configured to be coupled to a connector, wherein the connector is coupled to the medical device.

4. The endotracheal tube assembly of claim 2, wherein the medical device is a breathing medical device.

5. The endotracheal tube assembly of claim 2, wherein the distal end is beveled for easier insertion into the trachea.

6. The endotracheal tube assembly of claim 5, wherein the endotracheal tube further comprises an inflatable cuff disposed near the distal end of the endotracheal tube.

7. The endotracheal tube assembly of claim 6, wherein the wall of the endotracheal is configured with at least one murphy eye adjacent the distal end.

8. The endotracheal tube assembly of claim 2, wherein the tunnel is sealed at the distal end, the tunnel is having an opening at the proximal end.

9. The endotracheal tube assembly of claim 8, wherein the opening of the tunnel is configured to be closed for inserting into the trachea and opened for inserting the expander.

10. The endotracheal tube assembly of claim 1, wherein the tunnel is made of one or more layers of a stretchable material.

11. The endotracheal tube assembly of claim 10, wherein a joint between the tunnel and the wall is sealed by a stretchable lining.

12. A method of endotracheal intubation comprising a step of providing an endotracheal tube assembly, the endotracheal tube assembly comprising:
an endotracheal tube having a proximal end, a distal end, and a wall extending between the proximal end and the distal end, the proximal end configured to be couple to a medical device, the distal end configured to be inserted into a trachea;
a tunnel extending between the proximal end and the distal end of the endotracheal tube and interrupting the wall of the endotracheal tube, the tunnel configured to be expanded to increase the circumference of the endotracheal tube, the tunnel having an opening at the proximal end of the endotracheal tube; and
an expander having an elongated body, the expander configured to be removably received and retained into the tunnel, the expander configured to expand the tunnel, wherein a width of the expander is proportional to an increase in the circumference of the endotracheal tube.

13. The method of claim 12, wherein the method further comprises a step of inserting the endotracheal tube into the trachea.

14. The method of claim 13, wherein the method further comprises a step of inserting the expander of a first width into the tunnel.

15. The method of claim 14, wherein the opening of the tunnel is configured to be switched between an open position and a closed position, the method further comprises a step of opening the opening of the tunnel before inserting the expander.

16. The method of claim 14, wherein the method further comprises steps of:
removing the expander of first width from the tunnel; and
inserting the expander of second width into the tunnel, wherein the second width is different from the first width.

17. The method of claim 12, wherein the tunnel is made of a stretchable material.

18. The method of claim 12, wherein the tunnel is sealed at the distal end.

19. The method of claim 12, wherein one end of the expander is configured as a blunted tip, the blunted tip for insertion into the tunnel.
